(19) United States
(12) Patent Application Publication
(10) Pub. No.: US 2005/0238628 A1
(43) Pub. Date: Oct. 27, 2005

(54) Methods for treating cancer

(76) Inventor: Carl Anthony Blau, Seattle, WA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/100,817

(22) Filed: Apr. 7, 2005

Related U.S. Application Data
(60) Provisional application No. 60/560,557, filed on Apr. 8, 2004.

(51) Int. Cl. A61K 48/00

(52) U.S. Cl. 424/93.21

(57) ABSTRACT

The present invention relates to methods and compositions for treating cancer and related diseases.

### CROSS-REFERENCE TO RELATED APPLICATIONS

This application claims priority to provisional application Ser. No. 60/560,557 filed on Apr. 8, 2004 incorporated herein by reference in its entirety.

### FIELD

The present invention relates to methods and compositions for treating cancer and related diseases.

### BACKGROUND

Recombinant erythropoietin (EPO) is widely used for the treatment of cancer-related anemia. Several studies suggest, however, that erythropoietin may exert unanticipated negative effects in cancer patients (Brower, Nature Medicine 2003, 9(12):1439). One explanation for the possible adverse effects of EPO in cancer may reside in the finding that some non-hematopoietic cells carry EPO receptors (“EPO-Rs”). EPO-R expression has occasionally been observed in cancers arising from the kidney (Westenfelder et al., Kidney Int. 2000, 58(2):647-657), and because the kidney is also the primary site of EPO production, the potential for a paracrine loop has been noted. Brain (Juul et al., Pediatric Dev Pathol 1999, 2(2):148-158), breast (Juul et al., Pediatr Res 2000 48(5):660-667) and female genital tract tissues (Masuda et al., Int J Hematol 70:1-6, 2000, Acs et al., Am J Pathol. 2003 June; 162(6):1789-806) for example, have been shown to express EPO and its receptor. Several breast cancer cell lines express EPO-R and proliferate in response to EPO stimulation (ACS et al., Cancer Research 2001, 61(9):3561-3565). Reports suggest that many cancer cell types use the EPO system for growth and angiogenesis (Yasuda et al., Carcinogenesis 2003, 24(6):1021-1029).

In view of the foregoing, there exists a need for new cancer therapies that do not rely on erythropoietin administration. This invention meets this and other needs.

### DESCRIPTION OF THE DRAWINGS

FIG. 1 shows a graph of Ba/F3 cells capable of group in the presence of the CID, coumermycin.

FIG. 2 shows a graph of the percentage of coumermycin-responsive, HA-positive cells in cultures of mixed EpoR/abl-containing and GyrB/Flt3-containing Ba/F3 cells. The GyrB/Flt3 construct incorporates an HA tag. These results demonstrate a sharp reduction in erythropoietin responsive (HA-negative) cells in the presence of the CID, coumermycin.

### SUMMARY

The present invention relates, in part, to the discovery that blood cell production in cancer patients can be made independent of erythropoietin administration. The present invention provides, inter alia, methods of regulating the growth, including, e.g., differentiation and proliferation, of hematopoietic cells as well as methods of regulating blood cell production and methods of treating cancer in a mammal.

In accordance with some embodiments of the present invention, the provided methods for the regulation of blood cell production comprise the following steps: (i) identifying a mammal having a cancer that expresses or is suspected of expressing a receptor for a growth factor; (ii) introducing into the mammal a population of genetically modified hematopoietic cells comprising a recombinant nucleic acid construct encoding a fusion protein comprising at least one signaling domain and at least one ligand binding domain which is heterologous with respect to the signaling domain; and (iii) contacting the population of cells with a ligand, wherein the ligand binds to the ligand binding domain. Binding of a ligand to the ligand binding domain results in the dimerization or oligomerization of at least two of the fusion proteins thereby activating the signaling domain and initiating a signal for proliferation or differentiation of the population of cells. The proliferation or differentiation of the population of genetically engineered hematopoietic cells results in an increase in red blood cell levels in the mammal.

In some alternative embodiments, methods for the regulation of blood cell production comprise the following steps: (i) identifying a mammal having a cancer that expresses or is suspected of expressing a receptor for a growth factor; (ii) introducing into one or more hematopoietic cells of the mammal at least one recombinant nucleic acid construct encoding a fusion protein comprising at least one signaling domain and at least one ligand binding domain which is heterologous with respect to the signaling domain; and (iii) contacting the population of cells with a ligand, wherein the ligand binds to the ligand binding domain. Binding of a ligand to the ligand binding domain results in the dimerization or oligomerization of at least two of the fusion proteins thereby activating the signaling domain and initiating a signal for proliferation or differentiation of the population of cells. The proliferation or differentiation of the population of genetically engineered hematopoietic cells results in an increase in red blood cell levels in the mammal.

The present invention provides methods for treating cancer in a mammal. In accordance with some embodiments of the present invention, the provided methods comprise the following steps:(i) identifying a mammal having a cancer that expresses or is suspected of expressing a receptor for a growth factor; (ii) introducing into the mammal a population of genetically modified hematopoietic cells comprising a recombinant nucleic acid construct encoding a fusion protein comprising at least one signaling domain and at least one ligand binding domain which is heterologous with respect to the signaling domain; and (iii) contacting the population of cells with a ligand, wherein the ligand binds to the ligand binding domain. Binding of a ligand to the ligand binding domain results in the dimerization or oligomerization of at least two of the fusion proteins thereby activating the signaling domain and initiating a signal for proliferation or differentiation of the population of cells. The proliferation or differentiation of the population of genetically engineered hematopoietic cells results in an increase in red blood cell levels in the mammal.

In some alternative embodiments, methods for the treatment of cancer can comprise the following steps: (i) identifying a mammal having a cancer that expresses or is suspected of expressing a receptor for a growth factor; (ii) introducing into one or more hematopoietic cells of the mammal at least one recombinant nucleic acid construct encoding a fusion protein comprising at least one signaling domain and at least one ligand binding domain which is heterologous with respect to the signaling domain; and (iii) contacting the population of cells with a ligand, wherein the ligand binds to the ligand binding domain. Binding of a ligand to the ligand binding domain results in the dimerization or oligomerization of at least two of the fusion proteins thereby activating the signaling domain and initiating a signal for proliferation or differentiation of the population of cells. The proliferation or differentiation of the population of genetically engineered hematopoietic cells results in an increase in red blood cell levels in the mammal.

In some preferred embodiments, the methods of the present invention comprise an additional step of providing an anti-cancer agent to the mammal. In some embodiments, the anti-cancer agent is an erythropoietin antagonist.

In some embodiments, the growth factor is erythropoietin, granulocyte colony-stimulating factor, or granulocyte-macrophage colony stimulating factor.

The present invention also provides compositions for treating cancer in a mammal. The composition comprises a nucleic acid construct encoding a fusion protein comprising at least one signaling domain and at least one ligand binding domain which is heterologous with respect to the signaling domain, and a pharmaceutically acceptable carrier. Alternatively, the composition can comprise a population of genetically modified hematopoietic cells comprising the recombinant nucleic acid construct.

### DETAILED DESCRIPTION

It is widely known that the administration of growth factor, such as erythropoietin, to a mammal results in an increase in red blood cell levels in the mammal. It is now believed that certain cancers express growth factor receptors. Accordingly, it would be desirable to have alternative methods to increase blood cell levels that don't rely on the administration of a growth factor that may have the unwelcome effect of promoting or sustaining a cancerous state. The present invention provides such alternative methods.

The present invention provides, in some embodiments, methods for increasing blood cell levels in a mammal without providing an exogenous growth factor that is capable of binding to a receptor expressed by the cancer. In some embodiments, this presents a very effective way to treat anemia in patients who have cancers that express growth factor receptors. These methods utilize a recombinant nucleic acid construct encoding a fusion protein comprising at least one activation domain and at least one ligand binding domain which is heterologous with respect to the activation domain. Binding of a ligand to the ligand binding domain activates the activation domain resulting in the transduction of a signal for the proliferation or differentiation of cells transduced with the construct. By tightly regulating blood cell production in such a manner, administration of a growth factor that can promote the survival and/or proliferation of cancerous cells is rendered unnecessary. (For example, administration of erythropoietin to a mammal having a cancer that expresses an erythropoietin receptor or administration of granulocyte colony stimulating factor (“GCSF”) to a mammal having a cancer that expresses a GCSF receptor is rendered unnecessary.) Moreover, anti-cancer agents, including growth factor antagonists can be provided to the mammal. In some preferred embodiments, the present methods inhibit tumor growth, arrest tumor growth and/or cause the regression of tumors in a mammal while simultaneously treating anemia in the mammal.

The present methods generally involve the regulation or modulation of red blood cells. The success of the present methods does not depend on the correction or replacement of a gene that is defective in the subject. The nucleic construct can, however, contain additional genes. For example, in some embodiments, the nucleic acid construct will comprise a drug resistance gene that confers resistance to chemotherapy, e.g., dihydrofolate reductase or methylguanine methyltransferase.

Methods for pharmacologically regulated cell therapy using dimerization to initiate a signal for proliferation or differentiation of a population of genetically modified cells is known. See, for example, WO 99/34836, U.S. Pat. Nos. 5,741,899, 5,359,046, 5,869,337, 6,046,047, Neff and Blau, Blood, 97, 2535-2540, 2001, the disclosures of which are incorporated herein by reference in their entireties and for all purposes. The use of such a system in the context of the present invention, however, has not been known heretofore.

A target patient of the present invention is one who has a cancer that expresses or is suspected of expressing an endogenous growth factor receptor. A cancer that expresses an endogenous growth factor receptor can be one that only expresses the receptor in a small fraction of cancerous cells.

In an exemplary embodiment, of the present invention, to identify subject patients for treatment according to the methods of the invention, accepted screening methods can be employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to identify patients in need of therapy using the methods and formulations of the invention. In other embodiments, it will not be necessary to screen or examine a subject to identify the subject as one who can be treated using the disclosed methods, instead, all that will be necessary is for the subject to identify himself or herself as having cancer.

There are various means to diagnose cancer in a patient and to assess the efficacy of treatment using the methods of the present invention. Methods of diagnosing cancer in an individual can include a history and physical or neurological exam with particular attention to obvious lesions; palpable masses; ulcerations; swelling or enlargement of any masses or organs; erosion of bone; laterality, size and number of palpable lymph nodes; vision changes, focal deficit, tumor impingement on a specific nerve or structure; evidence of increased intracranial pressure; evidence of obstructive hydrocephalus. A diagnosis of cancer can be confirmed, for example, by imaging tests such as X-rays, nuclear scans and/or biopsies.

Methods known in the art for the detection of nucleic acids and proteins can be used for assessing whether cancerous tissue expresses a growth factor receptor, e.g., PCR, northern and Southern blots, dot blots, nucleic acid arrays, western blots, immunoassays such as immunoprecipitation, ELISA, proteomics assays, flow cytometry, immunohistochemistry, and the like (Yasuda et al., Carcinogenesis 2002, 123:11, 1797-1805; Yasuda et al., Carcinogenesis 2003, 24:6, 1021-1029; Liu et al., Oncogene 2004, 23, 981-990). Methods for producing polyclonal and monoclonal antibodies that react specifically with growth factor receptors are known to those of skill in the art. see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Antibodies for many growth factor receptors can be purchased from various companies, such as Research Diagnostics Inc. It is known that certain cancers express growth factor receptors. If a subject suffers from one of those cancers that are known to express growth factor receptors, it will not be necessary to further assess whether cancerous tissue obtained from the subject expresses a growth factor receptor. In those instances, it can be suspected that the cancer expresses a growth factor receptor and the methods of the present invention can be used for treatment. Cancers known to express growth factor receptors include, for example, renal cancer, uterine cancer, ovarian cancer, cervical cancer, liver cancer, brain cancer, breast cancer, colon cancer, CNS cancers, skin cancers, leukemia, and stomach cancer.

The term “growth factor receptor” as used herein denotes a cell-associated protein that binds to a growth factor. The interaction mediates the effect of the growth factor on the cell. A growth factor is a substance that mediates the proliferation and/or differentiation of cells. A hematopoietic growth factor is one that mediates the proliferation and/or differentiation of hematopoietic cells. Growth factor receptors include, but are not limited, to receptors for GCSF, GM-CSF, flt-3 (Fms-related Tyrosine kinase 3) ligand, stem cell factor, interleukins, such as IL-3, IL-6, IL-5, IL-7, IL-15, IL-21, IL-11 and IL-2, vascular endothelial growth factor, nerve growth factor, thrombopoietin, and erythropoietin.

“Cancer” refers to any of a number of diseases that are characterized by uncontrolled, abnormal proliferation of cells, as well as any of a number of characteristic structural and/or molecular features. A “cancerous cell” is understood as a cell having specific structural properties, lacking differentiation and in many instances, being capable of invasion and metastasis, see DeVita, V. et al. (eds.), 2001, Cancer Principles and Practice of Oncology, 6th. Ed., Lippincott Williams & Wilkins, Philadelphia, Pa.). The term cancer includes, but is not limited to, cancers of the female reproductive organs including, but not limited to, ovarian cancer, cervical cancer and uterine cancer; lung cancer; breast cancer; renal cell carcinoma; Hodgkin's lymphoma; Non-Hodgkin's lymphoma; cancers of the genitourinary system including, but not limited to, kidney cancer, prostate cancer, bladder cancer, and urethral cancer; cancers of the head and neck; liver cancer; cancers of the gastrointestinal system including, but not limited to, stomach cancer, esophageal cancer, small bowel cancer or colon cancer; cancers of the biliary tree; pancreatic cancer; cancers of the male reproductive system including, but not limited to, testicular cancer; Gestational trophoblastic disease; cancers of the endocrine system including, but not limited to, thyroid cancer, parathyroid cancer, adrenal gland cancer, carcinoid tumors, insulinomas and PNET tumors; sarcomas, including but not limited to, Ewing's sarcoma, osteosarcoma, liposarcoma, leiomyosarcoma, and rhabdomyosarcoma; mesotheliomas; cancers of the skin; melanomas; cancers of the central nervous system; pediatric cancers; and cancers of the hematopoietic system including, but not limited to all forms of leukemia, myelodysplastic syndromes, myeloproliferative disorders and multiple myeloma.

In some embodiments of the present invention, a subject treatable by the present methods will be suffering from a non-hematopoietic cancer that expresses or is suspected of expressing a receptor for an endogenous growth factor. A non-hematopoietic cancer is a cancer that occurs in a non-hematopoietic organ or tissue. Some embodiments will involve the use of autologous or allogeneic bone marrow or stem cell transplantation, however in other embodiments, a subject of the present invention is one that is not otherwise in need of bone marrow transplantation.

The methods of the present invention can be used to treat cancers that express or are suspected of expressing a receptor for an endogenous growth factor. The term “treating” or “treatment” refers to any indication of success in amelioration of an injury, pathology, or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. The term “treating” includes the administration of the construct or agents of the present invention to prevent or delay, to alleviate, or to arrest or inhibit development of the symptoms or conditions associated with cancer. For example, the term “treatment” can refer to the inhibition of tumor growth, the arrest of tumor growth, or the regression of already existing tumors. It can also refer to the alleviation of cancer-related anemia. The term “therapeutic effect” refers to the reduction, elimination, or prevention of the disease, symptoms of the disease, or side effects of cancer in the subject.

The term “subject” or “patient” as used herein means any mammalian patient or subject to which the compositions of the invention can be administered. The term “mammals” or “mammalian” includes human patients, as well as experimental animals such as, for example, non-human primates, rabbits, rats, dogs, cat, horses and mice, and other animals.

“Nucleic acid constructs,” as that term is used herein, denote nucleic acid molecules, e.g., DNA or RNA, used in the practice of this invention which are generally recombinant and which can exist in free form (i.g., not covalently linked to additional DNA or RNA) or can be present within a larger molecule such as a vector or a chromosome of a genetically engineered host cell. Nucleic acid constructs of particular interest are those which encode fusion proteins of this invention. The nucleic acid construct can further include one or more of the following elements relevant to regulation of transcription, translation, and/or other processing of the coding region or gene product thereof: transcriptional promoter and/or enhancer sequences, a ribosome binding site, introns, and the like.

“Recombinant,” “chimeric” and “fusion,” as those terms are used herein, denote materials comprising various component domains, sequences or other components which are mutually heterologous in the sense that they do not occur together in the same arrangement, in nature. More specifically, the component portions are not found in the same continuous polypeptide or nucleotide sequence or molecule in nature, at least not in the same cells or order or orientation or with the same spacing present in the chimeric protein or recombinant DNA molecule of this invention.

“Dimerization” as used herein refers to the association or clustering of two protein molecules, mediated by the binding of a ligand to a ligand binding domain of at least one of the proteins. In some embodiments, the dimerization is mediated by the binding of two protein molecules to a common divalent drug. The formation of a complex comprising two protein molecules, each of which containing one ligand binding domain e.g., a FKBP binding domain, together with a ligand which is divalent and binds to the FKBP binding domain (e.g., FK1012, AP1510 AP1903 or AP20187) is one example of such association or clustering.

“Oligomerization” and “multimerization” refer to the binding of more than two protein molecules, mediated by the binding of a ligand to at least two ligand binding domains on the proteins. In such instances, when one of the proteins contains more than one ligand binding domain, the presence of the ligand can lead to the clustering of more than two protein molecules. Embodiments in which the ligand is more than divalent (e.g., trivalent), its ability to bind to proteins bearing ligand binding domains also can result in clustering of more than two protein molecules. The formation of a tripartite complex comprising a protein containing at least one FRB domain, a protein containing at least one FKBP domain and a molecule of rapamycin is another example of such protein clustering. In certain embodiments of this invention, fusion proteins contain multiple ligand binding domains, such as, for example, multiple FRB and/or FKBP domains. Complexes of such proteins can contain, for example, more than one molecule of rapamycin or a derivative thereof or other dimerizing agent and more than one copy of one or more of the constituent proteins. Again, such multimeric complexes are still referred to herein as tripartite complexes to indicate the presence of the three types of constituent molecules, even if one or more are represented by multiple copies. The formation of complexes containing at least one divalent ligand and at least two protein molecules can be referred to as “dimerization,”“oligomerization,” “multimerization,” “clustering” or “association.

“Divalent,” as that term is applied to ligands in this document, denotes a ligand which is at least divalent with respect to proteins containing a binding domain which binds to the ligand. Said differently, a divalent drug is capable of complexing with at least two protein molecules which contain ligand binding domains, effectively cross-linking the proteins to form a three (or greater number)-component complex. The term multivalent as used herein includes divalent ligands.

The term “erythropoietin” refers to any polypeptide or protein that has the biological activity of human erythropoietin, including erythropoietin analogs, erythropoietin isoforms, erythropoietin mimetics, erythropoietin fragments, hybrid erythropoietin proteins, fusion proteins oligomers and multimers of the above, glycosylation pattern variants of the above, and muteins of the above. Specific examples of erythropoietin include, Epoetin alfa (EPREX®, ERYPO®, and PROCRIT®); darbepoietin (Aranesp®), novel erythropoiesis stimulating protein (NESP) (a hyperglycosylated analog of recombinant human erythropoietin (Epoetin) described in European patent application EP640619); human erythropoietin analog-human serum albumin fusion proteins described in the international patent application WO9966054; erythropoietin mutants described in the international patent application WO9938890; erythropoietin omega, which may be produced from an Apa I restriction fragment of the human erythropoietin gene described in U.S. Pat. No. 5,688,679; altered glycosylated human erythropoietin described in the international patent application WO991178; and PEG conjugated erythropoietin analogs described in WO9805363 or U.S. Pat. No. 5,643,575.

“Genetically engineered cells” denotes cells which have been modified by the introduction of recombinant or heterologous nucleic acids (e.g., one or more DNA constructs or their RNA counterparts) and further includes the progeny of such cells which retain part or all of such genetic modification. A population of cells refers to more than one cell. In embodiments wherein a population of genetically modified cells is introduced into a mammal, the population of cells can be allogeneic, autologous, or syngeneic with respect to the mammal.

“Transduction” and “transducing” refer to any manner of delivery of nucleic acids into cells, including, but not limited to, transformation, transfection, electroporation and infection.

The term “differentiation,” as used herein, refers to an alteration in the pattern of gene expression in cells which typically is associated with one or more of the following: changes in morphology, lineage, motility, adhesion, cell cycle regulation, and the like.

Any hematopoietic cell type can be used in the practice of this invention, as long as it can be engineered to express the fusion proteins and induced to grow, proliferate and/or differentiate upon dimerization or oligomerization. These include, among others, cells obtained from embryonic, juvenile or adult mammals, including stem cells, progenitor cells and precursor cells of various types and tissues. A variety of such cells and methods for obtaining and handling them are known in the art. By way of non-limiting example, such cells include stem cells, progenitor cells and precursor cells from bone marrow, peripheral blood, cord blood or fetal liver (e.g., hematopoietic stem cells and lymphoid, myeloid and erythroid precursor cells). Hematopoietic cells also include, for example, common lymphoid progenitor cells, T cells (e.g., helper, cytotoxic, and suppressor cells), B cells, plasma cells, natural killer cells, common myeloid-progenitor cells, monocytes, macrophages, mast cells, leukocytes, basophils, neutrophils, eosinophils, megakaryocytes, platelets, and erythroid cells

A “hematopoietic stem cell” (HSC) refers to a population of cells capable of both self-renewal and differentiation into all defined hematopoietic lineages, i.g., myeloid, lymphoid or erythroid lineages. HSCs are capable of repopulating the hematopoietic system of a recipient who has undergone myeloablative treatment. HSCs can ultimately differentiate into hematopoietic cells including without limitation, common lymphoid progenitor cells, T cells (e.g., helper, cytotoxic, and suppressor cells), B cells, plasma cells, natural killer cells, common myeloid-progenitor cells, endothelial cells, monocytes, macrophages, mast cells, leukocytes, basophils, neutrophils, eosinophils, megakaryocytes, platelets, and erythroid cells. HSCs can be identified, for example, by the presence of cell surface antigens of primitive phenotypes.

The term “lineage committed cell” refers to a stem cell that is no longer pluripotent but has become restricted to only a subset of lineages, e.g., in the case of hematopoietic stem cells, e.g.,cells restricted to the erythroid, erythroid-megakaryocyte, megakaryocyte, granulocyte-macrophage, granulocyte, macrophage, granulocyte-erythroid macrophage-megakaryocytic, megakaryocytic lineages. Lineage restricted cells include common lymphoid progenitors and common myeloid progenitors (Kondo et al., Ann Rev Immunol 21:759-806. 2003). The lineage committed cell subsequently differentiates to specialized cell types, e.g., in the case of hematopoietic lineage committed cells, to cell types such as, for example, erythrocytes or neutrophils.

In some preferred embodiments of the present invention, the term “hematopoietic cells” as used herein refers to either red blood cells or other cells capable of differentiation into red blood cells, including hematopoietic stem cells, bone marrow cells, cord blood cells, and peripheral blood cells.

Binding domains of the present invention can be extracellular or intracellular. The term “extracellular ligand binding domain” refers to the portion of a fusion protein of the present invention which is outside of the plasma membrane of a cell and binds to at least one extracellular ligand. The extracellular ligand binding domain can include, for example, the extracytoplasmic portion of a transmembrane protein, a cell surface or membrane associated protein, a secreted protein, a cell surface targeting protein, or a cell adhesion molecule. After binding of the extracellular ligand binding domain with a ligand, two or more fusion proteins will become associated with each other by dimerization or oligomerization. The term “intracellular binding domain” refers to the portion of the fusion protein which is inside of the plasma membrane of a cell than binds to at least one intracellular ligand. After binding of the intracellular ligand binding domain with a ligand, two or more fusion proteins will become associated with each other by dimerization or oligomerization.

The signaling domain refers to any domain capable of modulating growth, proliferation, or differentiation of the genetically modified hematopoietic cells upon oligomerization or dimerization of the fusion proteins. Signaling domains can include, for example, receptor cytoplasmic domains, including domains comprising naturally occurring human peptide sequence, as well as fragments, subunits and analogs of the foregoing which retain one or more of the characteristic biological activities of the parent protein, e.g., induction of cellular growth, proliferation and/or differentiation. Signaling domains can also include transcription factors. When this invention is applied to human patients, it is preferred that the signaling domain comprise a naturally occurring human peptide sequence.

Signaling domains that can be used in the present invention include, for example, the cytoplasmic signal-transducing domains of the cytokine/hematopoietin receptor superfamily. The members of this mammalian receptor superfamily can initiate proliferative signals in a wide variety of cell types. These receptors are structurally related to each other. The cytoplasmic domains of the signal-transducing subunits can contain conserved motifs that are critical for transduction of proliferative signals (Bazan, Current Biology, 1993, 3:603-606; Boulay and Paul, Current Biology, 1993, 3:573-581; Wells, Current Opinion in Cell Biology, 1994, 6:163-173; Sato and Miyajima, Current Opinion in Cell Biology, 1994, 6:174-179; Stahl and Yancopoulos, Cell, 1993, 74:587-590, Minami et al., Ann. Rev. Immunol., 1993, 11:245-267; Kishimoto et al., Cell, 1994, 76:253-262).

Many cytokine and growth factor receptors associate with common β subunits that interact with tyrosine kinases and/or other signaling molecules. Such cytokines and growth factor receptors can be used as cytoplasmic signaling domains in fusion proteins of this invention. These include, for example, receptors for GM-CSF, IL-3 and IL-5 which contain a common signal transducing or β chain which has a large cytoplasmic domain whose membrane proximal region is critical for c-myc induction and proliferative signaling activity; receptors for IL-6, CNTF (ciliary neurotrophic factor), LIF (leukemia inhibitory factor), OSM (oncostatin M), and IL-11 which have a common signal transducing chain, gp130 (glycoprotein 130: the common subunit for the receptors for IL-6, leukemia inhibitory factor, and oncostatin M), with a cytoplasmic domain whose membrane proximal region is critical for signaling activity; receptors for IL-2, IL-4, IL-7, IL-9, IL-13, IL-15 which share IL-2γ. Receptors for IL-2 and IL-15 also share a IL-2β transducing component which is homologous to the cytoplasmic domain of the G-CSF receptor.

Additional signal-transducing components of the cytokine receptors that can be used as signaling domains of the present invention include, but are not limited to, EPO-R (erythropoietin receptor), G-CSFR (granulocyte colony stimulating factor receptor), GM-CSFRα (granulocyte macrophage colony stimulating factor receptor α), GM-CSFRβ, GHR (growth hormone receptor), PRLR (prolactin receptor), IFNRα/β (interferon α/β receptor), IFNRγ, TFR (tissue factor receptor), and TPOR (thrombopoietin or mpl-ligand receptor).

Examples of receptor tyrosine kinases that can be used as signaling domains of the present invention are tyrosine kinases of subclass I, including but not limited to EGF-R(epidermal growth factor receptor), ATR2/neu, HER2/neu, HER3/c-erfaB-3, and Xmrk; tyrosine kinases of subclass II including, but not limited to, insulin-R, IGF-l-R [insulin-like growth factor receptor], IRR; tyrosine kinases of subclass III including, but not limited to PDGF-R(platelet derived growth factor receptor)-A, PDGF-R-B, CSF-l-R (Macrophage colony stimulating factor-receptor/c-Fms), c-kit (stem cell factor receptor), flt-3, and STK-1/Flk-2; and tyrosine kinases of subclass IV including, but not limited to fibroblast growth factor receptor-1 (FGFR-1), FGFR-2, FGFR-3, FGFR-4, vascular endothelial growth factor receptors, Tie family receptors, and neurotrophic tyrosine kinases including, but not limited to, Trk family, includes NGF-R, Rorl,2 (Schlessinger, Cell 103:211-225, 2000).

Receptors which associate with tyrosine kinases upon oligomerization or dimerization can also be used as signaling domains of the present invention. These include members of the CD3 ζ and CD3 η family (found primarily in T cells, associates with Fyn); β chains of Fcε R1 (found primarily in mast cells and basophils); γ chains of Fcγ RIII/CD16 (found primarily in macrophages, neutrophils and natural killer cells); CD3 γ, -δ, and -ε (found primarily in T cells); Ig-α/MB-1 and Ig-β/B29 (found primarily in B cells).

The proliferation signaling domains employed in constructing the constructs of the present invention can also be obtained from any member of the Janus kinase or JAK eukaryotic family of tyrosine kinases, including Tyk2, JAK1, JAK2, JAK3 and Ptk-2. Members of the Janus kinase family are found in all cell types. They associate with various signal transducing components of the cytokine receptor superfamily discussed above and respond to the binding of extracellular inducer by the phosphorylation of tyrosines on cytoplasmic substrates. The proliferation signaling domains employed in constructing the constructs of the present invention can also be obtained from any member of the Raf family (Raf-1, A-Raf, B-Raf) or the MAP (mitogen activated protein kinase) eukaryotic family of serine threonine kinases (Schaeffer and Weber, Mol. Cell. Biol. 1999, 19:2435-2444; Steelman et al., Leukemia 2004, 18:189-218).

The proliferation signaling domains employed in constructing the constructs of the present invention can also be obtained from any member of the Signal transducers and activators of transcription (STAT) transcription factor family, including STAT-1, STAT-2, STAT-3, STAT-4, STAT-5a, STAT-5b, and STAT-6 (Calo et al., J. Cell Phys 2003, 197:157-168, 2003, Steelman et al., Leukemia 2004, 18:189-218, 2004).

The proliferation signaling domains employed in constructing the constructs of the present invention can also be obtained from any member of the phosphatidylinositol kinase family, PI3-kinase and Akt (Steelman et al., Leukemia 2004, 18:189-218, 2004).

In some embodiments, fusion proteins of this invention can be targeted to the membrane by, for example, incorporating a myristoylation sequence, e.g., from c-src, or any other membrane targeting or anchoring sequence into the fusion protein's design. In other embodiments, signal transduction can be induced by directing the subcellular localization of a signaling molecule. For example, MAP kinase signaling can be induced by bringing a Raf-1-E. coli DNA gyrase B (GyrB) oligomer to the cell membrane using coumermycin (Farrar et al., Nature 383:178-181, 1996).

The signaling domain, as it exists naturally or as it may be truncated, modified or mutated, can be at least about 10, usually at least about 30 amino acids, more usually at least about 50 amino acids, and generally not more than about 500 amino acids, usually not more than about 200 amino acids. (See Romeo, et al. Cell, 1992, 68:889). While any species can be employed, the species endogenous to the host cell is usually preferred.

In some embodiments of the present invention, several signaling domains, such as for example EpoR and mpl, GCSFR and mpl, or GCSFR and EpoR can be used in combination to create novel composite signaling domains. Alternatively, for a receptor which requires more than a single chain for signaling, such as the interleukin 2 (IL-2) receptor, a construct can be used in which the component chains are fused together. Additionally, the cells can provided with more than one chimeric protein, each of which binds a different ligand. For example, in some embodiments, a construct encoding a first fusion protein containing at least one FRB domain and a flt-3 domain and a second fusion protein containing at least one FKBP domain and a c-kit domain can be used. In these cells, the c-kit containing proteins will homodimerize upon addition of FK1012, while the c-kit and flt-3 proteins will heterodimerize upon the addition of rapamycin. In a similar approach, DNA encoding a “daisy chain” of two or more ligand binding domains targeted to the membrane using, e.g., a myristoylation site, can be used in the present methods. Along with this construct, any number of constructs encoding different ligand-binding domain/signaling domain fusions can be introduced. Using this approach, a multiplicity of proliferative responses can be achieved upon contact with one or more ligands.

In some embodiments, fusion proteins of this invention can contain a cytoplasmic domain from one of the various cell surface membrane receptors, including mutants thereof, wherein activation of the receptor induces cellular proliferation.

The nucleic acid constructs of the present invention can further comprise a transmembrane domain. Transmembrane domains of the present invention can be contributed by the protein contributing the signaling domain, the protein contributing the extracellular binding domain, or by a totally different protein. Typically, the transmembrane domain will be naturally associated with one or the other of the domains. In some embodiments, the transmembrane domain of the ζ, η, or FcεFR1γ chains or related proteins which contain a cysteine residue capable of disulfide bonding, are used so that the resulting chimeric protein will be able to form disulfide linked dimers with itself, or with unmodified versions of the ζ, η, or FcεFR1γ chains or related proteins. In some embodiments, the transmembrane domain is selected or modified by amino acid substitution to avoid binding of such domains to the transmembrane domains of the same or different surface membrane proteins to minimize interactions with other members of the receptor complex. In other embodiments, the transmembrane domain of ζ, η, or FcεFR1-γ chains and -β, MB1 (Ig α), B29 (Igβ), Bovine Leukemia Virus gp30 (BLV gp30), or CD3-γ, δ, or ζ is used in order to retain physical association with other members of the receptor complex.

The nucleic acid constructs of the present invention comprise ligand binding domains. The ligand binding domains can be any protein domain for which a ligand is known or can be identified, wherein upon binding of the ligand to the ligand binding domain, the signaling domain is activated thereby initiating a signal for proliferation or differentiation. The binding domain can be internal (intracellular) or external (extracellular) to the cellular membrane depending on the nature of the construct and choice of the ligand. The ligand binding domains can be obtained from the binding domains of a variety of proteins. Of particular interest are binding proteins for which ligands (preferably small organic ligands) are known or may be readily produced. These ligand binding domains include the FKBPs and cyclophilin receptors, DNA gyrase B (Farrar et al., Nature 383:178-181, 1996), the estrogen and progesterone receptors, and the like, as well as receptors that can be obtained from antibodies, particularly the heavy or light chain subunit, mutated sequences thereof, random amino acid sequences obtained by stochastic procedures, combinatorial syntheses, and the like. In preferred embodiments, the receptor domains will be at least about 50 amino acids, and fewer than about 350 amino acids, usually fewer than 200 amino adds, either as the natural domain or truncated active portion thereof. Preferably the binding domain will be small (for example less than about 25 kD, to allow efficient transfection in viral vectors), monomeric, nonimmunogenic, and will have synthetically accessible, cell permeable, nontoxic ligands that can be configured for dimerization. Also preferably, the intracellular ligand binding domain will not bind substantially to any endogenous ligands, but will bind only to ligands exogenously administered. For dimerization-based approaches to regulation of cellular functions in human subjects, the use of fusion proteins which contain protein domains of human origin, or derivatives thereof, are preferred.

The ability to employ in vitro mutagenesis or combinatorial modifications of sequences encoding proteins allows for the production of libraries of proteins which can be screened for binding affinity for different ligands. For example, the present invention contemplates totally randomizing a sequence of 1 to 5, 10 or more codons, at one or more sites in a DNA sequence encoding a binding protein, making an expression construct and introducing the expression construct into a unicellular microorganism to develop a library. The library can be subsequently screened for binding affinity to one or desirably a plurality of ligands. The best affinity sequences which are compatible with the cells into which they would be introduced can then be used as the binding domain. The ligand can be screened with the host cells to be used to determine the level of binding of the ligand to endogenous proteins. A binding profile can be defined weighting the ratio of binding affinity to the mutagenized binding domain with the binding affinity to endogenous proteins. Those ligands which have the best binding profile can then be used as the ligand. Phage display techniques, as a non-limiting example, can be used in carrying out the foregoing.

The extracellular ligand binding domain can be obtained from any wide variety of extracellular domains of eukaryotic transmembrane proteins, secreted proteins, or other proteins associated with ligand binding and/or signal transduction. The extracellular ligand binding domain can be part of a protein which is, for example, monomeric, homodimeric, heterodimeric, or associated with a large number of proteins in a non-covalent or disulfide-bonded complex. For example, the extracellular ligand binding domains can comprise monomeric or dimeric immunoglobulin molecules or portions or modifications thereof. Diabodies can be used as extracellular ligand binding domains of the present invention. The monomeric or dimeric immunoglobulin molecules or portions or modifications thereof and diabodies can be prepared as described in U.S. Pat. No. 5,741,899 incorporated herein by reference in its entirety and for all purposes.

Naturally occurring receptors can also be used as extracellular binding domains of the present invention, including cell differentiation antigens such as CD4 and CD8, cell surface proteins expressed in lymphocytes such as CD20 or CD25, cytokine or hormone receptors or cell adhesion molecules. The receptor may be responsive to a natural ligand, an antibody or fragment thereof, a synthetic molecule, e.g., drug, or any other agent which is capable of initiating a signal. In addition, the receptor-binding domains of soluble protein ligands or portions thereof can be employed as intracellular ligand binding domains as well as binding portions of antibodies, cytokines, hormones, or serum proteins. Additionally, the soluble components of the cytokine receptors, including, for example, IL-6R, IL-4R, and IL-7R can be used (Boulay and Paul Current Biology 1993, 3: 573-581).

In some embodiments, the ligand binding domain can be a binding site for an antibiotic, and the antibiotic can serve as the ligand (for example, GyrB and coumermycin).

“Hybrid” extracellular ligand binding domains can also be used in the present invention. For example, two or more antigen-binding domains from antibodies of different specificities, two or more different ligand-binding domains, or a combination of these domains can be connected to each other by oligo- or polypeptide linkers to create multispecific extracellular binding domains. These intracellular ligand binding domains can be used to create the constructs of the present invention which can respond to two or more different extracellular ligand molecules.

In some embodiments, where a receptor is a molecular complex of proteins, and only one chain has the major role of binding to the ligand, only the extracellular portion of the ligand binding protein will be used. In other embodiments, where the extracellular portion can complex with other extracellular portions of other proteins or form covalent bonding through disulfide linkages, dimeric or multimeric extracellular regions can be formed. In some embodiments, the entire extracellular region will not be required and truncated portions thereof can be employed, provided that the truncated portion is functional.

In some embodiments, a few amino acids at the joining region of the natural protein domain can be deleted, usually not more than about 30, more usually not more than about 20. In some embodiments, a small number of amino acids can be introduced at the borders, usually not more than about 30, more usually not more than about 20. The deletion or insertion of amino acids will usually be as a result of the needs of the construction, providing for convenient restriction sites, ease of manipulation, improvement in levels of expression, proper folding of the molecule or the like. In addition, one or more amino acids can be substituted with a different amino acid for similar reasons, usually not substituting more than about five amino acids in any one domain.

Typically, the signal sequence at the 5′ terminus of the open reading frame which directs the chimeric protein to the surface membrane will be the signal sequence of the intracellular ligand binding domains. However, in some embodiments, this sequence can be exchanged for a different signal sequence. Since the signal sequence will be removed from the protein during processing, the particular signal sequence is not critical to the subject invention.

The intracellular ligand binding domain can be obtained from any wide variety of intracellular domains of a variety of intracellular proteins. For example, eukaryotic steroid receptor molecules can be used as intracellular ligand binding domain (e.g. the receptors for estrogen, progesterone, androgens, glucocorticoids, thyroid hormone, vitamin D, retinoic acid, 9-cis retinoic acid and ecdysone). In addition, variants of steroid and other receptors which fail to bind their native inducer, but still bind to an antagonist, can be used. For example, a C-terminal deletion mutant of the human progesterone receptor, which fails to bind progesterone, can be clustered by the addition of progesterone antagonists, including RU 486 (Wang et al., Proc Natl Acd Sci 1994, 91: 8180-8184). Binding domains from the eukaryotic immunophilin family of molecules can also be used as the intracellular ligand binding domain. Examples include but are not limited to members of the cyclophilin family: mammalian cyclophilin A, B and C, yeast cyclophilins 1 and 2, Drosophila cyclophilin analogs such as ninaA; and members of the FK506-binding protein (FKBP) family: the various mammalian isoforms of FKBP and the FKBP analog from Neurospora (Schreiber, Science, 1991, 251:283-287; McKeon, Cell, 1991, 66:823-826; Friedman and Weissman, Cell, 1991, 66:799-806; Liu et al., Cell, 1991, 66:807-815). For example, the ligand binding domain of the immunophilin, FKBP12, which can be clustered in the cytoplasm by the addition of FK1012, a synthetic dimeric form of the immunosuppressant FK506 (Spencer et al., Science 262:1019-1024 (1993) can be used as an intracellular ligand binding domain.

In some embodiments, the ligand binding domains are drug binding domains and are based on, for example, FKBP12, and in some cases, the FRB domain of FRAP (“FKBP-rapamycin-associated protein”). Those domains can be engineered to recognize novel FKBP ligands and/or rapamycin derivatives, e.g., as disclosed in PCT/US94/01617 and PCT/US96/09948 (WO 96/41865).

Depending on design preferences of the practitioner, a wide variety of drugs can be used as ligands. FK1012, cyclosporin-based divalent ligands, fujisporin and related types of semisynthetic ligands are disclosed in WO 94/18317 and PCT/US94/08008 (WO 95/02694). Drugs based on synthetic FKBP ligand monomers are disclosed in WO 96706097 and WO 97/31898, and drugs based on rapamycin and derivatives are disclosed in WO 96/41865. All of the foregoing components may be used in the practice of this invention and the full contents of the various documents referred to above are incorporated herein by reference. Those documents also provide guidance in the design of constructs encoding such chimeras, expression vectors containing them, design and use of suitable target gene constructs, and their use in engineering host cells. As further guidance in that regard, specific examples are provided below which illustrate the design, construction and use of constructs for the regulated expression of target genes using dimerization of signal transduction domains.

FKBP, FRB, cyclophilin and other drug binding domains comprising naturally occurring peptide sequence can be used in the design of fusion proteins for use in practicing this invention. Alternatively, domains derived from naturally occurring sequences but containing one or more mutations in peptide sequence, generally at up to 20 to 10 amino acid positions, and preferably at 1-5 positions, more preferably at 1-3 positions and in some cases at a single amino acid residue, can be used in place of the naturally occurring counterpart sequence and can confer a number of important features.

For example, illustrative mutations-of current interest in FKBP domains include the following:

TABLE 1

| | | | |
|---|---|---|---|
| F36A | Y26V | F46A | W59A |
| F36V | Y26S | F48H | H87W |
| F36M | D37A | F48L | H87R |
| F36S | I90A | F48A | F36V/F99A |
| F99A | I91A | E54A | F36V/F99G |
| F99G | F46H | E54K | F36M/F99A |
| Y26A | F46L | V55A | F36M/F99G |

The entries in TABLE 1 identify the native amino acid by single letter code and sequence position, followed by the replacement amino add in the mutant. Thus, F36V designates a human FKBP12 sequence in which phenylalanine at position 36 is replaced by valine. F36V/F99A indicates a double mutation in which phenylalanine at positions 36 and 99 are replaced by valine and alanine, respectively.

Illustrative FKB mutations, especially for use with rapamycin analogs bearing substituents other than —OMe at the C7 position include amino acid substitutions for one of more of the residues Tyr2038, Phe2039, Thr2098, Gln2099, Trp2101 and Asp2102. Exemplary mutations include Y2038H, Y2038L, Y2038V, Y2038A, F2039H, F2039L, F2039A, F2039V, D2102A, T2098A, T2098N, and T2098S. Rapamycin derivatives bearing substituents other than —OH at C28 and/or substituents other than ═O at C30 can be used to obtain preferential binding to FRAP proteins bearing an amino acid substitution for Glu2032. Exemplary mutations include E2032A and E2032S. Peptide sequence numbering and rapamycin numbering is with reference to WO 96/41865.

Illustrative mutations in cyclophilin domains (and corresponding cyclosporin compounds) are disclosed in WO 94/18317 and may also be adapted for use in practicing the subject invention. Another illustrative examples of a drug-protein interaction is the interaction between coumermycin and DNA gyrase B (Farrer et al., 1996).

Nucleic acid constructs can be designed in accordance with the principles, illustrative examples and materials and methods disclosed in the patent documents and scientific literature cited herein, each of which is incorporated herein by reference, with modifications and further exemplification as described herein. Components of the constructs can be prepared in conventional ways, where the coding sequences and regulatory regions can be isolated, as appropriate, ligated, cloned in an appropriate cloning host, analyzed by restriction or sequencing, or other convenient means. Particularly, using PCR, individual fragments including all or portions of a functional unit can be isolated, where one or more mutations can be introduced using “primer repair;” ligation, in vitro mutagenesis, and the like, as appropriate. In the case of DNA constructs encoding fusion proteins, DNA sequences encoding individual domains and sub-domains can be joined such that they constitute a single open reading frame encoding a fusion protein capable of being translated in cells or cell lysates into a single polypeptide harboring all component domains. The DNA construct encoding the fusion protein can then be placed into a vector that directs the expression of the protein in the appropriate cell type(s). Alternatively, the desired DNA constructs can be generated by homologous recombination in bacteria using commercially available techniques that are well described in the literature (Zhang et al., Nature Biotechnology 18 (2000) 1314-1317). Accordingly, fusion proteins of the present invention can be generated by homologous recombination into endogenous gene loci. For biochemical analysis of the encoded chimera, it can be desirable to construct plasmids that direct the expression of the protein in bacteria or in reticulocyte-lysate systems. For use in the production of proteins in mammalian cells, the protein-encoding sequence can be introduced into an expression vector that directs expression in these cells. Expression vectors suitable for such uses are well-known in the art. Various sorts of such vectors are commercially available.

Any means for the introduction of heterologous DNA into mammalian cells, human or non-human, can be adapted to the practice of this invention. Conventional viral and non-viral based gene transfer methods can be used to introduce the constructs of the invention in mammalian cells or target tissues. Such methods can be used to administer the recombinant constructs to cells in vitro. In some embodiments, the recombinant constructs are administered for in vivo or ex vivo gene therapy uses. Non-viral vector delivery systems include, for example, DNA plasmids, naked nucleic acid, and nucleic acid complexed with a delivery vehicle such as a liposome. Viral vector delivery systems include DNA and RNA viruses, which can have episomal or integrated genomes after delivery to the cell. For a review of gene therapy procedures, see Anderson, Science 1992, 256:808-813; Nabel & Felgner, TIBTECH 1993, 11:211-217; Mitani & Caskey, TIBTECH 1993, 11:162-166; Dillon, TIBTECH 1993, 11:167-175; Miller, Nature 1992, 357:455-460; Van Brunt, Biotechnology 1988, 6(10):1149-1154; Vigne, Restorative Neurology and Neuroscience 1995, 8:35-36; Kremer & Perricaudet, British Medical Bulletin 1995, 51(1):31-44; Haddada et al., in Current Topics in Microbiology and Immunology Doerfler and Bohm (eds) (1995); and Yu et al., Gene Therapy 1994, 1:13-26.

Methods of non-viral delivery of recombinant constructs of the invention include, for example, lipofection, microinjection, biolistics, virosomes, liposomes, immunoliposomes, polycation or lipid:nucleic acid conjugates, naked DNA, artificial virions, and agent-enhanced uptake of DNA. Lipofection is described in, for example, U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 1995, 270:404-410; Blaese et al., Cancer Gene Ther. 1995, 2:291-297; Behr et al., Bioconjugate Chem. 1994, 5:382-389; Remy et al., Bioconjugate Chem. 1994, 5:647-654; Gao et al., Gene Therapy 1995, 2:710-722; Ahmad et al., Cancer Res. 1992, 52:4817-4820; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of recombinant constructs encoding fusion proteins of the invention can take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of constructs of the invention include retrovirus, lentivirus, human foamy virus, adenovirus, adeno-associated virus (AAV), adeno-AAV, and herpes simplex virus vectors for gene transfer. Viral vectors are currently the most efficient and versatile method of gene transfer in target cells and tissues. Integration in the host genome is possible with the retrovirus, human foamy virus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Pseudotypes that are well suited for the transduction of human hematopoietic cells can include the envelopes of gibbon ape leukemia virus (GaLV) (Horn et al., Blood 2002; 100:3960-7) and endogenous feline leukemia virus (RD114) (Neff et al., Mol. Ther. 2004; 9:157-9). Virus production can be achieved using murine or human packaging cell lines. Lentivirus vectors and human foamy virus vectors are retroviral vectors that are able to transduce or infect non-dividing cells and typically produce high viral titers. Selection of a retroviral gene transfer system would therefore depend on the target tissue. Retroviral vectors are generally comprised of cis-acting long terminal repeats with packaging capacity for up to 6-10 kb of foreign sequence. The minimum cis-acting LTRs are sufficient for replication and packaging of the vectors, which are then used to integrate a construct into the target cell to provide permanent transgene expression. Widely used retroviral vectors include those based upon murine leukemia virus (MuLV), Simian Immuno deficiency virus (SIV), human immuno deficiency virus (HIV), human foamy virus and combinations thereof (see, e.g., Buchscher et al., 1992, J. Virol. 66:2731-2739; Johann et al., 1992, J. Virol. 66:1635-1640; Sommerfelt et al., Virol. 1990, 176:58-59; Wilson et al., J. Virol. 1989, 63:2374-2378; Mergia and Heinkelein, Curr Top Microbiol Immunol. 2003; 277:131-59. Miller et al., J. Virol. 1991, 65:2220-2224; PCT/US94/05700).

Methods for using oncoretrovirus, lentivirus or human foamy virus vectors for transfer of the fusion protein of the present invention into hematopoietic cells, including hematopoietic stem cells, are well described in the literature (reviewed in Brenner and Malech, Biochim Biophys Acta. 2003; 1640:1-24). Hematopoietic stem cells can be obtained by isolating mononuclear cells from the bone marrow or from the peripheral blood, the latter most commonly done using leukapheresis. In most cases, collection of peripheral blood mononuclear cells is performed following several days of G-CSF administration, which acts to mobilize stem cells from the bone marrow to the blood. Hematopoietic stem cells can be enriched from mononuclear cell collections using either positive selection systems (most commonly based on the expression of CD34) or negative selection systems, resulting in the depletion of cells expressing lineage specific cell surface markers. Populations enriched in stem cells can then be subjected to gene transfer. In the case of oncoretrovirus vectors, hematopoietic cells undergo a period of “prestimulation”, during which they are cultured in the presence of a combination of growth factors (usually including stem cell factor, IL-6, thrombopoietin, and flt-3 ligand), most commonly for a period of 48 hours. Gene transfer is commonly accomplished by preloading retrovirus supernatant on retronectin-coated dishes, and then culturing the cells in retrovirus supernatant in the presence of the same or similar combination of cytokines as used during the prestimulation step. Cultures in the presence of retroviral supernatant are typically performed over a period of 48 hours, with 2 or more changes of retroviral supernatant during the culture period. In contrast to oncoretroviral vectors, gene transfer using lentivirus or human foamy virus vectors can commonly be performed overnight without added growth factors. The fewer ex vivo manipulations associated with use of lentivirus or human foamy virus vectors may improve the engraftability of hematopoietic stem cells transduced with these vectors.

Transduced hematopoietic stem cells can have an engraftment defect following transplantation. While many myeloablative conditioning regimens have been described these are encumbered by toxicity, and it is desireable to employ treatments that facilitate the engraftment of transduced hematopoietic stem cells while minimizing toxicity to the patient. A number of attenuated conditioning regimens that faciliate the engraftment of autologous or allogeneic donor stem cells, have been devised (reviewed in Georges and Storb, Int J Hematol. 2003; 77:3-14). These include the administration of fludarabine and low doses of radiation therapy (typically 200 cGy) (Maris M, Storb R. Immunol Res. 2003; 28(1):13-24). Busulfan administration has been used successfully to faciliate the engraftment of transduced autologous hematopoietic stem cells. (Aiuti et al., Int J Hematol. 2003 January; 77(1):3-14). Additionally, cells can be genetically modified or otherwise treated to facilitate their engraftment, for example by inhibiting the function of the surface membrane protein, CD26 (Christopherson et al., Science. 2004 August; 305(5686):1000-3).

In applications where transient expression of the fusion protein of the invention is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with the recombinant constructions, (see, e.g., West et al., Virology 1987, 160:38-47; U.S. Pat. No. 4,797,368; WO 93/24641; Kotin, Human Gene Therapy, 1994, 5:793-801; Muzyczka, J Clin. Invest. 1994, 94:1351 (1994). Construction of recombinant AAV vectors are described in a number of publications, including U.S. Pat. No. 5,173,414; Tratschin et al., Mol. Cell. Biol. 1985, 5:3251-3260; Tratschin, et al., Mol. Cell. Biol. 1984, 4:2072-2081; Hermonat & Muzyczka, PNAS 1984, 81:6466-6470; and Samulski et al., J. Virol. 1989, 63:03822-3828.

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following are examples of proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (see Table 1 in Kotin, Human Gene Therapy 1994, 5:793). A transgene incorporating the various constructs of this invention can similarly be included in an AAV-based vector. As an alternative to inclusion of a constitutive promoter such as CMV to drive expression of the recombinant DNA encoding the fusion protein(s), an AAV promoter can be used (ITR itself or AAV p5 (Flotte, et al. J. Biol. Chem. 1993, 268:3781).

Such a vector can be packaged into AAV virions by reported methods. For example, a human cell line such as 293 can be co-transfected with the AAV-based expression vector and another plasmid containing open reading frames encoding AAV rep and cap under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, Current Opinion in Biotechnology 1992, 3:533; Kotin, Human Gene Therapy 1994, 5:793). Methods to improve the titer of AAV can also be used to express the transgene in an AAV vinon. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Additionally, the efficiency of AAV transduction can be increased by treating the cells with an agent that facilitates the conversion of the single stranded form to the double stranded form, as described in Wilson, et al. WO96/39530. AAV vectors have been used to direct homologous recombination so that genes can be modified at their endogenous loci (Hirata et al., Nat Biotechnol. 2002 July; 20(7):735-8). Using this or other approaches for homologous recombination, novel proteins can be generated, for example, by inserting sequences encoding the ligand binding domain directly adjacent to endogenous genetic sequences encoding a signaling domain of interest. Alternatively, in some embodiments, sequences encoding a desired signaling domain can be inserted adjacent to an endogenously expressed ligand binding domain.

Concentration and purification of the virus can be achieved by reported methods such as banding in cesium chloride gradients, as was used for the initial report of AAV vector expression in vivo (Flotte, et al. J. Biol. Chem. 1993, 268:3781) or chromatographic purification, as described in O'Riordan, et al. WO97/08298.

For additional detailed guidance on AAV technology which can be useful in the practice of the subject invention, including methods and materials for the incorporation of a transgene, the propagation and purification of the recombinant AAV vector containing the transgene, and its use in transfecting cells and mammals, see e.g., U.S. Pat. Nos. 4,797,368; 5,139,941; 5,173,414; 5,252,479; 5,354,678; 5,436,146; 5,454,935; 5,658,776 and WO 93/24641.

pLASN and MFG-S are examples are retroviral vectors that have been used in clinical trials (Dunbar et al., Blood 1995, 85:3048-305; Kohn et al., Nat. Med. 1995, 1:1017-102; Malech et al., PNAS 1997, 94:22 12133-12138). PA317/pLASN was the first therapeutic vector used in a gene therapy trial. (Blaese et al., Science 1995, 270:475-480). Transduction efficiencies of 50% or greater have been observed for MFG-S packaged vectors. (Ellem et al., Immunol Immunother. 1997, 44(1):10-20; Dranoff et al., Hum. Gene Ther. 1997, 1:111-2.

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 1998, 351:9117 1702-3, Kearns et al., Gene Ther. 1996, 9:748-55).

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiply types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 1998, 7:1083-9. Additional examples of the use of adenovirus vectors for gene transfer in clinical trials include Rosenecker et al., Infection 1996, 24:1 5-10; Sterman et al., Hum. Gene Ther. 1998, 9:7 1083-1089; Welsh et al., Hum. Gene Ther. 1995, 2:205-18; Alvarez et al., Hum. Gene Ther. 1997, 5:597-613; Topf et al., Gene Ther. 1998, 5:507-513; Sterman et al., Hum. Gene Ther. 1998, 7:1083-1089.

Packaging cells can be used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 1995, 92:9747-9751, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, often after selection for cells which have incorporated the vector.

Ex vivo cell transfection for research or for gene therapy (e.g., via re-infusion of the transfected cells into the host organism) is well known to those of skill in the art. In some embodiments, cells are isolated from the subject organism, transfected with a recombinant construct encoding a fusion protein of the invention, and re-infused back into the subject mammal (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, hematopoietic stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Stem cells are isolated for transduction and differentiation using known methods. For example, stem cells are isolated from bone marrow cells by standard immunomagnetic methods using antibodies that deplete differentiated cell types or that positively select for stem cell antigens such as CD34 or CD 133. panning the bone marrow cells with antibodies which bind unwanted cells, such as CD4+ and CD8+ (T cells), CD45+ (panB cells), GR-1 (granulocytes), and Iad (differentiated antigen presenting cells) (see Huntenburg et al., J Hematother. 1998 7:175-83).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Generally, the DNA or viral particles are transferred to a biologically compatible solution or pharmaceutically acceptable delivery vehicle, such as sterile saline, or other aqueous or non-aqueous isotonic sterile injection solutions or suspensions, numerous examples of which are well-known in the art, including Ringer's, phosphate buffered saline, or other similar vehicles.

Preferably, the DNA or recombinant virus is administered in sufficient amounts to transfect cells at a level providing therapeutic benefit without undue adverse effects.

Optimal dosages of DNA or virus depends on a variety of factors as discussed herein and may thus vary somewhat from patient to patient. Again, therapeutically effective doses of viruses are generally considered to be in the range of about 1×10⁵ to about 1×10¹⁰ pfu of virus/ml.

The present invention provides methods of regulating the production of genetically modified hematopoietic cells as well as method for treating cancer in a mammal. The methods of the present invention include a step of contacting a population cells with a ligand. In some embodiments, the contacting step is effected by administering to the mammal a ligand capable of binding to the ligand binding domain of the fusion protein. In other embodiments, the ligand is endogenously expressed in the mammal and thus will naturally contact the ligand binding domain. The ligand can be provided in vivo or ex vivo.

The ligand can be administered as desired using pharmaceutically acceptable materials and methods of administration. Depending upon factors such as the binding affinity of the ligand, the response desired, the manner/route of administration, the biological half-life and bioavailability of the ligand, and the number of engineered cells present, various protocols known in the art can be employed.

In some embodiments of the present invention, in addition to providing genetically modified cells to a mammal and regulating such cells in the mammal by ligand binding, an additional therapeutic agent is concomitantly administered to the mammal. The additional therapeutic agent can be any compound or composition that can be used to treat cancer, i.g., an anti-cancer agent.

“Concomitant administration” means administration of the additional therapeutic agent at such time that both the additional therapeutic agent and the binding of the ligand to the ligand binding domain of a construct of the present invention will have a therapeutic effect. Such concomitant administration may involve concurrent (i.e. at the same time), prior, or subsequent administration of the therapeutic agent in relation to administration of the construct or ligand. A person of ordinary skill in the art, would have no difficulty determining the appropriate timing, sequence and dosages of administration for particular drugs, ligands, and constructs of the present invention.

One example of an anti-cancer agent that can be administered is an erythropoietin antagonist. Examples of erythropoietin antagonists include, but are not limit to the erythropoietin mutant R103A (Bums et al., Blood. 2002 Jun. 15; 99(12):4400-5), the EPO mimetics EMP and EMP33 (Johnson et al., Biochemistry, 1998, 37, 3699-1710; Livnah et al., Nat Struct Biol. 1998, 5(11):993-1004; Yasuda et al., Carcinogenesis 2003, 24:1021-1029), anti-EPO antibodies (Yasuda et al., Carcinogenesis. 2002, 23(11):1797-805), and the soluble form of the EPO receptor (Yasuda et al., Carcinogenesis. 2002, 23(11):1797-805).

Therapeutic drugs useful for treating cancer in combination therapy with the methods of the present invention include, for example, chemotherapeutic agents, alone, or in combination with, radiation treatment, surgical treatment, or treatments using biological or immunomodulatory agents. Chemotherapeutic drugs useful in treating cancer include alkylating agents, antimetabolites, natural products, hormones and antagonists (reviewed in B. A. Chabner and D. L. Longo Eds. Cancer Chemotherapy and Biotherapy, 3^{rd} Edition, 2001). These include, for example, nitrogen mustards, including but not limited to mechlorethamine, cyclophosphamide, ifosfamide, melphalan, and chlorambucil; ethylenimines and methylmelamines, including but not limited to, hexamethylmelamine and thiotepa; alkyl sulfonates, including but not limited to, busulfan, carmustine, lomustine, semustine, and streptozocin; triazenes, including but not limited to, dacarbazine and temozolamide, folic acid analogs, including but not limited to, methotrexate and trimetrexate 5-fluoropyrimidines including but not limited to, fluorouracil, floxuridine, ftorafur, capecitabine, and eniluracil,cytidine analogs, including cytarabine; 5-azacytidine, gemcitabine, purine analogs and related inhibitors, including but not limited to, mercaptopurine, thioguanine, fludarabine, cladribine, and pentostatin; vinca alkaloids, including but not limited to, vinblastine, and vincristine; taxanes including paclitaxel and docetaxel, topoisomerase II inhibitors, including but not limited to, etoposide, amsacrine and teniposide; topoisomerase I targeting agents including, but not limited to camptothecin, topotecan, irinotecan, and karenitecin,antibiotics, including but not limited to, dactinomycin, daunorubicin, doxorubicin, bleomycin, plicamycin, and mitomycin; enzymes, including but not limited to, L-Asparaginase; biological response modifiers, including but not limited to, IL-2, interferon-alfa IL-1, IL-2, IL-4, IL-12, tumor necrosis factor and macrophage colony stimulating factor, platinum coordination complexes, including but not limited to, cisplatin, oxaloplatin, and carboplatin; anthracenediones, including but not limited to, mitoxantrone; thalidomide and derivatives including, but not limited to revemid, proteosome inhibitors including, but not limited to bortezomib, substituted ureas, including but not limited to, hydroxyurea; methylhydrazine derivatives, including but not limited to, procarbazine; adrenocortical suppressants, including but not limited to, mitotane and aminoglutethimide; adrenocorticosteroids, including but not limited to, prednisone; progestins and dexamethasone, including but not limited to, hydroxyprogesterone caproate, medroxyprogesterone acetate, and megestrol acetate; estrogens, including but not limited to, diethylstilbestrol and ethinyl estradiol; antiestrogens, including but not limited to, tamoxifen; androgens, including but not limited to, testosterone propionate and fluoxymesterone; antiandrogens, including but not limited to, flutamide; and gonadotropin releasing hormone analogs, including but not limited to, leuprolide, aromatase inhibitors including but not limited to anastrazole (brand name Arimidex®), exemestane (brand name Aromasin®), and letrozole (brand name Femara®); antibodies directed against cell surface molecules including but not limited to rituximab, trastuzumab, CAMPATH, cetuximab and bevacizumab, including antibodies conjugated to toxins, including but not limited to gemtuzumab, and antibodies conjugated to radioisotopes including, but not limited to ibritumomab; anti-cancer antibodies that have been humanized to avoid the development of human antimouse antibodies; small molecule tyrosine kinase inhibitors including, but not limited to, gleevec and iressa (reviewed in Noble et al., Science. 2004; 303:1800-5); faranesyl transferase inhibitors including, but not limited to R115777 (tipifamib, Zarnestra®), SCH66336 (lonafamib, Sarasar®) and BMS-214662, including formulations of chemotherapy drugs including, but not limited to, liposomal formulations, including arsenic trioxide, including cancer differentiating agents including but not limited to all trans retinoic acid, including cancer treatments that use adoptive immunotherapy and including cancer treatments that use gene therapy. Methods of administering chemotherapeutic agents for treating cancer are known in the art. (Goodman and Gilman's The Pharmacological Basis of Therapeutics, Ninth Edition).

For treatment purposes, the ligands and/or anti-cancer agents disclosed herein can be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). The ligands and/or anti-cancer agents can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection).

In this context, a therapeutically effective dosage of the ligand is a dose that is effective to increase blood cell production in a mammal e.g., red blood cell production. In certain embodiments, the mammal will be suffering from cancer-related anemia and the dose will be effective to treat anemia in the patient. For example, the dose will be effective to increase a mammal's hemoglobin levels to about 10 g/dL or greater. In certain embodiments, the dose will be effective to treat cancer in the patient. A change in red blood cells can be routinely assessed using standard clinical tests known in the art conducted serially within a patient over time. Such tests include, for example, measurements of hemoglobin, hematocrit, and red cell number. A change in serum erythropoietin levels can be readily detected, using standard serial tests of erythropoietin levels in a patient over time.

In healthy patients, serum levels of erythropoietin generally range from about 11 to about 19 mu/ml. There is an inverse relationship between the hematocrit and the circulating erythropoietin level. As the hematocrit falls, the erythropoietin level in the blood rises. In a particularly preferred embodiment, the ligand dose will be effective to decrease and/or suppress circulating endogenous erythropoietin levels in the subject. Accordingly, in some embodiments, the amount of ligand provided to a mammal is adjusted such that erythropoietin levels are reduced to fall within or below the normal range.

Methods of assessing erythropoietin hemoglobin and erythropoietin levels in a mammal are known in the art and are thus not described herein in detail. A therapeutically effective dose of an anti-cancer agent is a dose that is effective to treat cancer in the patient.

Determination of effective dosages of ligands and/or anti-cancer agents is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response). In alternative embodiments, an “effective amount” or “therapeutically effective dose” of the biologically active agent(s) will simply inhibit or enhance one or more selected biological activity(ies) correlated with a disease or condition, as set forth above, for either therapeutic or diagnostic purposes.

The actual dosage of biologically active agents will of course vary according to factors such as the extent of exposure and particular status of the subject (e.g., the subject's age, size, fitness, extent of symptoms, susceptibility factors, etc), time and route of administration, as well as other drugs or treatments being administered concurrently. Dosage regimens can be adjusted to provide an optimum prophylactic or therapeutic response. By “therapeutically effective dose” herein is meant a dose that produces effects for which it is administered. More specifically, a therapeutically effective dose of the compound(s) of the invention preferably alleviates symptoms, complications, or biochemical indicia of diseases associated with cancer, including cancer-related anemia. The exact dose will depend on the purpose of the treatment, and will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

The ligands and/or anti-cancer agents of the present invention can be provided in a pharmaceutical pack or kit comprising one or more containers filled with one or more of the ingredients of the pharmaceutical compositions. Optionally associated with such containers can be a notice in the form prescribed by a governmental agency regulating the manufacture, use or sale of pharmaceutical or biological products, which notice reflects approval by the agency of manufacture, use or sale for human administration.

The following examples of specific embodiments for carrying out the present invention are offered for illustrative purposes only, and are not intended to limit the scope of the present invention in any way.

The disclosures of all publications, patents and patent applications cited herein are hereby incorporated by reference in their entirety and for all purposes.

### EXAMPLES

### Example 1

### Experiments in Erythropoietin (Epo)-Deficient Mice

Steps:

a. Cell Lines.

Cells will be implanted subcutaneously, intravenously, intramuscularly, intraperitoneally, intrathecally, or retroorbitally into mice. One or more of a number of human or murine cell lines can be used. Desirable cell lines include those that express the EpoR endogenously, or are engineered to express the EpoR or a derivative of the EpoR. Ideal cell lines are those that exhibit epo-responsiveness in vitro or in vivo. Epo-responsiveness is demonstrated by Epo-responsive cell division or Epo-responsive cell survival under a variety of culture conditions including serum deprivation, deprivation of other growth factors, upon exposure to conditions that induce apoptosis including chemotherapeutic agents or irradiation, or by the activation of Epo-responsive signaling pathways upon exposure to erythropoietin. Cell lines that fulfill some or all of these criteria include, but are not limited to, Ba/F3 cells engineered to express an EpoR/abl fusion protein (Okuda et al., 1997, J Clin Invest 100:1708-1715), SCH, HepG2, HMV1, G361, P39, 220, DLD1, A549, SBC3, Hela, PC-3 (Yasuda et al., 2003), MCF-7, BT-549, T47D, MDA-134, MDA-231, Hep3B, LHSY5Y, U87, U251, U373 (Acs et al., 2001, Cancer Research 61:3561-3565), K562, ACHN, or Caki 1 (Liu et al., Oncogene, 2004, Oncogene 23:981-990), neuroblastoma cell lines CHLA-90, SK-N-RA, KCNR, LHN, CHLA-15, CHLA-20, SK-N-FI, SK-N-BE-2, CHLA-171, SAN, LAN-5, LAN-6, and CHLA-134, Ewing's sarcoma family of tumor cell lines CHP-100, A5838, SK-N-MC, TC-106, and TC-71, the adult breast cancer cell line MCF-7, the colon cancer cell line HT-29, the glioma cell lines T98G and A172 (Batra et al., Lab Invest 2003:83:1477-1487), and the TEL/AML1 ALL tumor cell line REH (Matsuo and Drexler, Leuk Res. 1998; 22:567-79). Mouse tumor cell lines will be implanted into isogenic or congenic mouse strains. Immune deficient mice can be used for the implantation of immunologically disparate tumors or tumor cell lines of mouse or human origin.

A number of nonerythroid cell lines have been examined for EpoR expression. It was found that Reh cells, a human TEL/AML1⁺ ALL line (Rosenfeld et al., Nature. 1977; 267:841-3, Uphoff et al Leukemia. 1997; 11:441-7.) expressed significant levels of EpoR, and exhibit STAT5 phosphorylation upon the addition of erythropoietin.

b. Generating Recipients for Transplantation of F36Vmpl Transduced Marrow Cells and Implantation of Tumor Cells

Mice that have little or no capacity to produce erythropoietin will be used as recipients for both of F36Vmpl transduced marrow cells and the Epo responsive tumor cells described above. Homozygous Epo null (Epoko) mice die at approximately 13 days of gestation due to profound anemia (Wu et al., Cell 1995. 83:59-67). An alternative mouse model is one that has a transgene insertion into the EpoR gene (Epotg) resulting in a markedly reduced capacity to make Epo. The erythropoietin-SV40 T antigen (Epo-TAgh) transgenic mouse has a targeted disruption in the 5′ untranslated region of the EPO gene that dramatically reduces expression such that the homozygous animal, although viable, is severely anemic with a hematocrit of approximately 13.2%+/−3.3% (Maxwell et al., Kidney Int. 1993; 44:1149-1162). This mouse has served as a model for erythropoietin gene therapy (Binley et al., Blood. 2002 Oct. 1; 100(7):2406-13). To eliminate immune mediated rejection of subsequently implanted tumor cells, the heterozygous Epoko or either heterozygous or homozygous EpoTAgh mice will be extensively backcrossed (typically at least ten backcrosses) into an immune deficient mouse background (nude, SCID, NOD-SCID, NOD-SCID-beta2 microglobulin ko, or other immune deficient background). Homozygous Epoko or Epotg mice will be generated by heterozygote matings. Since homozygous Epoko mice die prenatally, in utero injections of erythropoietin or in utero transfusions of isogenic rbcs will be performed to generate live homozygote births. Following birth, the red blood cell counts of either Epoko or Epo-TAgh mice will be maintained in the normal range through intermittent injections of erythropoietin.

c. Transplanting F36Vmpl-Transduced Marrow Cells.

Donor marrow will be obtained from mice generated in (b). Marrow cells from donors 8-16 weeks of age will be transduced with a retroviral vector encoding F36Vmpl using approaches that are well described in the literature (Jin et al., PNAS, 1998, 95:8093-8097; Jin et al., Nature Genetics, 2000, 26:64-66; Richard et al., Blood, 2004, 103:4432-9). Transduced marrow cells will then be transplanted into the recipients generated in (b). In cases where recipient mice are immune competent, myeloablative doses of radiation (1050 cGy) will be used. Since immune deficient mice have an exaggerated sensitivity to radiation, sublethal doses (typically 350 cGy) will likely be required for these recipients.

d. Following transplantation of F36Vmpl-transduced marrow cells, red cell production in a subset of the mice will be induced by treatment with a synthetic dimerizer such as AP1903 (Clackson et al., 1998 Proc Natl Acad Sci 1; 95(18):10437-42.), AP20187 (Jin et al., 2000, supra) or others. A typical course of dimerizer administration is 10 mg/kg/day intraperitoneally for 3 days every 4 weeks (Jin et al., 2000, supra Richard et al., 2004, supra), however the dose, schedule and route of dimerizer administration will be adjusted based on serial measurements of red cell counts and hematocrit, with a goal of achieving red cell counts or hematocrits that approach or are within the normal range. In mice that retain some capacity to produce endogenous erythropoietin, it may be desirable to suppress endogenous Epo production by driving circulating numbers of red cells to levels that surpass the normal range. In the remaining mice, similar levels of circulating red cells will be achieved using intermittent subcutaneous injections of erythropoietin (20 mcg/kg or 180 mcg/kg determined by peptide mass without carbohydrate) or darbepoietin (10 mcg/kg or 30 mcg/kg determined by peptide mass without carbohydrate) (Hartley et al., Br J Haematol. 2003; 122:623-36.). The dose and schedule of erythropoietin or darbepoietin administration will be adjusted either upward or downward based on serial measurements of red cell number and/or hematorcrit. Mice will be assigned to treatment with either dimerizer or Epo/darbepoietin beginning concomitant with the transplantation of F36Vmpl transduce marrow cells, or alternatively, all mice are treated with erythropoietin/darbepoietin immediately post transplantation and then divided into the dimerizer and Epo/darbopoietin groups several weeks to months post transplantation.

e. Injection of Tumor Cells.

After generating the 2 groups of mice (either dimerizer-dependent or Epo-dependent) as described in (d), both groups will be injected with an EpoR expressing tumor cell line or primary tumor tissue as described in (a). Tumor cells will be injected either concomitant with the transplantation of transduced marrow cells, or after hematological recovery following marrow transplantation. 10⁵-10⁷ tumor cells will be injected intravenously, intramuscularly, subcutaneously, intraperitoneally, retroorbitally or intrathecally. Alternatively, fragments of primary tumors will be implanted intramuscularly, subcutaneously or intraperitoneally.

f. Endpoints.

Endpoints will be measurements of tumor cell mass and survival. Mice will be monitored daily to evaluate their general health status, tumor status, red cell number and survival. In addition, mice in both groups will continue to receive intermittent injections of dimerizer or Epo to maintain red cell numbers in a similar range between the 2 groups. Tumor cell mass will be measured directly by 3 dimensional measurements of palpable or visible masses, or by indirect measurements of tumor cell markers (for example, carcinoembryonic antigen, alpha fetoprotein, CA12.5) in the blood. Alternatively, tumor cell mass will be measured by a variety of imaging studies including radiographic studies, nuclear medicine studies, or bioluminescent imaging (Jenkins et al., 2003a, Jenkins et al., 2003b). In addition, tumor cell mass will be assessed at autopsy in all mice. Alternatively mice in both groups will be sacrificed and tumor cells in all organs will be compared between the two groups by gross morphology and histologically, or by creating cell suspensions from each organ and quantifying absolute numbers of tumor cells using tumor specific antibodies and flow cytometry. The tumor burden in mice treated with dimerizer is expected to be discernibly reduced compared to the tumor burden of erythropoietin-treated mice. The tumor burden of dimerizer versus erythropoietin-treated mice will be compared by direct palpation, by measuring the size of tumors visible macroscopically at autopsy, by flow cytometry, or by measurement of microscopic tumors upon examining tissue sections. Alternatively, tumor burden can be assessed by non-invasive imaging, by measuring levels of circulating tumor cell markers, or by comparing survival between dimerizer-and epo-treated mice.

### Example 2

### Inhibition of Hemopoietic Cells that Ectopically Express a Full-Length Epo-Receptor or EpoR Derivative, In Vivo

Marrow cells from donor mice (C57BL/6, B6D2F1 or other strains of mice) will be divided into 2 halves. One half of the cells will be transduced with an F36Vmpl vector, while the other half of cells will be transduced with a vector that encodes full length Epo receptor (Lacout et al., Exp Hematol 1996 24:18-25). Alternatively, the remaining half of cells will be transduced with a vector that encodes an EpoR-Abl fusion protein (Okuda et al., J Clin Invest 1997, 100:1708-1715), or a truncated EpoR (Kirby et al., Blood, 2000, 95:3710-3715). Transductions will be performed using standard techniques. The 2 vectors will be distinguishable on the basis of markers that can be discriminated by flow cytometry. For example, the F36Vmpl vector will be tagged with a green fluoroescent protein reporter, whereas the EpoR vector will be tagged with a dsRed reporter. Marrow cells transduced with the F36Vmpl^{GFP} and EpoR^{dsRed} will be mixed in equal proportion and transplanted into lethally irradiated congenic recipients. Following transplantation, the frequency of GFP versus dsRed expressing cells in the marrow and peripheral blood (determined by flow cytometry) will be monitored over time. After establishing a stable baseline, the Epo-responsiveness of the dsRed-positive population will be demonstrated by administering Epo or darbepoietin to half of the mice. AP20187 or AP1903 will be administered at a dose of 10 mg/kg/day for 3-7 days every 2 to 4 weeks in the remaining half of mice. It is expected that a CID treatment will induce a decline in EpoR containing, dsRed-positive cells, demonstrating that CID treatment can reduce the number of cells expressing the EpoR.

### Example 3

### Inhibition of Cells with Tumorgenic Potential, In Vitro, Using AP1903 or AP20187

Ba/F3 cells expressing F36Vmpl^{GFP} and EpoR^{dsRed} or EpoR-Abl^{dsRed} (Okuda et al., 1997, supra) will be generated in the presence of IL3, then IL3 will be washed away, and cells expressing either GFP or dsRed will be mixed in equal proportion and cultured in the presence of CID (AP20187 or AP1903 100 nM) or in the presence of Epo. CID exposure is expected to produce an expansion of GFP positive cells and a loss of dsRed positive cells. Alternatively, Epo exposure is expected to expand dsRed positive cells with a loss of cells expressing GFP.

### Example 4

### Inhibition of Cells with Tumorgenic Potential in Non-Epo Deficient Mice

Tail vein injection of Ba/F3EpoRAbl cells into nude mice followed by injections of erythropoietin has been shown to induce death with a timecourse that is dependent on the concentration of exogenously administered Epo (Okuda et al., 1997, supra). Of note, Ba/F3 cell tumor cell models have also been reported by injecting Ba/F3 cells into Balb-C mice (Tse et al., Leukemia. 2000, 14(10):1766-76.). Nude or Balb-C mice will be transplanted with marrow cells transduced with the F36Vmpl^{GFP} vector as described previously (Jin et al., 2000 supra). Reduced intensity irradiation may be required due to enhanced radiosensitivity in these mouse strains. Following hematological recovery, complete blood counts and the frequency of genetically modified red cells will be measured in each mouse by flow cytometry. Approximately 3-4 months post transplantation, after achieving stable level of gene transfer, half of the mice will receive CID (typically AP20187 10 mg/kg IP×3-5 days every 2-4 weeks), whereas the remaining mice will provide non-CID treated controls. 4 weeks after initiating CID treatment, all mice will begin a program of routine phlebotomies, with withdrawal of 200 microliters of blood per week. Red cell counts, hematocrit and erythropoietin levels will be monitored weekly between the two groups. The schedule of CID administration and schedule/volume of phlebotomies will be adjusted to maintain a significantly higher red cell count and hematocrit and lower Epo level in the CID treated group relative to the control mice. To maintain red cell counts within the desired range it may be necessary to supplement phlebotomies with intermittent administration of phenylhydrazine, or to use phenylhydrazine injections alone as a means for inducing a chronic stable hemolytic anemia. After achieving stable differences in hematocrit, red cell count and Epo levels between CID and control groups, all mice will receive tail vein injections of 5×10⁶-1×10⁷ BaF3 EpoR-Abl cells. Mice will continue to receive monthly injections of either CID or no drug and differences in survival between the 2 groups would be monitored. Additional endpoints would be the number of Ba/F3 EpoR-Abl cells in the blood, marrow, spleen and other organs and determined histologically and by flow cytometry. A modification of this experiment would be to add intermittent injections of exogenous erythropoietin to mice in the non-CID treated mice in order to maintain equivalent red cell numbers between the two groups. Dimerizer-treated mice are expected to have a longer survival or fewer Ba/F3 EpoR-Abl cells in the blood, marrow, spleen or other organs compared to control mice.

### Example 5

### Incorporating the Use of Anti-Epo Peptides or Blocking Antibodies

Experiments using Epo-blocking peptides or antibodies with the models developed in examples 1 and 4. The rationale for using Epo-blocking strategies in the Epo-deficient model described in example 1 is that some tumors and tumor cell lines express Epo, creating the potential for an autocrine loop, with the tumor providing its own source of Epo production.

Epo-blocking approaches will be tested by modifying the experiment described in example 1 as follows. Mice in whom hematocrits are maintained in a normal or supernormal range with dimerizer treatment will receive implants/injections of EpoR expressing primary tumors or tumor cell lines. Half of the mice will be treated with an erythropoietin antagonist such as EMP33 (Livnah et al., supra) EMP9 (Johnson et al., 1998, supra; Yasuda et al., 2003, supra), a blocking anti-EpoR antibody (for example R2) (Yasuda et al., 2002, supra), or soluble EpoR (Nagao et al., 1992, supra; Yasuda et al., 2002, supra). While these reagents will initially be administered as described (Yasuda et al., 2003, supra), doses and schedules will be adjusted as needed to maximize their antagonistic effects. In some cases, different antagonists may be combined. Treatment with dimerizer and the Epo antagonist(s) will continue for periods ranging from between one and sixteen weeks. Mice treated with the epo antagonists are expected to exhibit a longer survival, or a reduced tumor cell mass as measured either directly by palpation, by macroscopic examination, by microscopic evaluation, or by measuring surrogate tumor cell markers.

### Example 6

### Inhibition of Cells with Tumorgenic Potential, In Vitro, Using Coumermycin

Generating Ba/F3 Cells Expressing a EpoR/Ablfusion:

A cDNA containing the EpoRIAbl construct was used to transduce Ba/F3 cells by retroviral gene transfer. After two days, IL-3 containing medium was washed away, and cells were cultured in the presence of erythropoietin (3 units/ml), without IL-3, to generate a polyclonal pool of erythropoietin dependent cells.

Generating Ba/F3 Cells Expressing a Coumerymin Inducible Derivative of Flt-3.

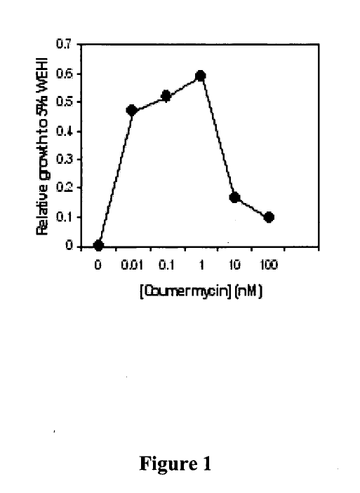

An MSCV-based vector was constructed that encodes a fusion protein containing a myristylation domain linked to DNA gyrase B, which in turn is linked to the intracellular portion of murine flt-3, which in turn is linked to an HA epitope tag. This vector, designated GyrB/Flt3, was used to generate Ba/F3 cells that were capable of growth in the presence of the CID, coumermycin (see FIG. 1). Ba/F3 cells expressing the GyrB/Flt3 construct can be distinguished from Ba/F3 cells expressing EpoR/Abl using intracellular flow cytometry with an antibody directed against the HA tag.

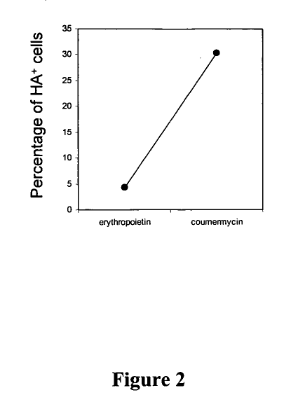

Ba/F3 cells expressing EpoR/Abl (hence referred to as EpoR/Abl cells) were mixed with Ba/F3 cells expressing GyrB/Flt3 and cultured in the absence of IL-3, plus either erythropoietin (3 U/ml) or coumermycin (1 nM). After 4 days, the frequency of cells expressing the HA tag was determined by flow cytometry. The results demonstrated a sharp reduction in erythropoietin responsive (HA-negative) cells in the presence of CID (FIG. 2).

1. A method for treating cancer in a mammal comprising:
(i) identifying a mammal having a cancer that expresses or is suspected of expressing a receptor for a growth factor;
(ii) introducing into said mammal a population of genetically modified hematopoietic cells, said population of cells comprising a recombinant nucleic acid construct encoding a fusion protein comprising at least one signaling domain and at least one ligand binding domain which is heterologous with respect to the signaling domain, wherein binding of a ligand to the ligand binding domain results in the dimerization or oligomerization of at least two of said fusion proteins thereby activating the signaling domain and initiating a signal for proliferation or differentiation of said population of cells, wherein the proliferation or differentiation of said population of genetically engineered hematopoietic cells results in an increase in red blood cell levels in said mammal; and
(iii) contacting said population of cells with a ligand, wherein said ligand binds to the ligand binding domain.

2. The method of claim 1, wherein the proliferation or differentiation of said population of genetically engineered hematopoietic cells treats anemia in said mammal.

3. The method of claim 1, wherein the ligand binding domain is an antibody and activation of the signaling domain occurs upon binding of an antigen to the antibody.

4. The method of claim 1, wherein the ligand binding domain comprises a naturally occurring cell surface protein that is not expressed by the cancer.

5. The method of claim 1, wherein the ligand binding domain is a cell differentiation antigen or a receptor that is not expressed by the cancer.

6. The method of claim 1, wherein the ligand binding domain is a drug binding domain, the ligand is a drug that is exogenously provided to the mammal, and the drug is multivalent and binds to two or more fusion protein molecules thereby activating the activation domain.

7. The method of claim 1, wherein the population of cells are treated with the ligand ex vivo.

8. The method of claim 1, wherein the population of cells are treated with the ligand in vivo.

9. The method of claim 1, wherein the signaling domain comprises some or all of the cytoplasmic portion of a receptor for a growth or differentiation factor.

10. The method of claim 1, wherein the signaling domain comprises some or all of c-kit, gp130, flt-3, the growth hormone receptor, EGF, FGF, CSF-1, G-CSF, thrombopoietin, erythropoietin, granulocyte macrophage colony stimulating factor, prolactin, or hepatocyte growth factor.

11. The method of claim 1, wherein the signaling domain comprises at least part or all of a Janus tyrosine kinase.

12. The method of claim 1, wherein the signaling domain comprises at least part or all of a MAP kinase.

13. The method of claim 1, wherein the signaling domain comprises at least part or all of a STAT transcription factor.

14. The method of claim 1, wherein the signaling domain comprises at least part or all of a phosphatidylinositol kinase.

15. The method of claim 1, wherein the cells contain an additional heterologous RNA or DNA construct.

16. The method of claim 1 further comprising the step of providing an anti-cancer agent to the mammal.

17. A method for treating cancer in a mammal comprising:
(i) identifying a mammal having a cancer that expresses or is suspected of expressing a receptor for an endogenous growth factor;
(ii) introducing into one or more hematopoietic cells at least one recombinant nucleic acid construct encoding a fusion protein comprising at least one signaling domain and at least one ligand binding domain which is heterologous with respect to the signaling domain, wherein binding of a ligand to the ligand binding domain results in the dimerization or oligomerization of at least two of said fusion proteins thereby activating the signaling domain and initiating a signal for proliferation or differentiation of said cells, wherein the proliferation or differentiation of said population of genetically engineered hematopoietic cells results in an increase in red blood cell levels in said mammal; and
(iii) contacting said population of cells with a ligand, wherein said ligand binds to the ligand binding domain.

18. The method of claim 17 further comprising providing an anti-cancer agent to the mammal.

19. A method for the regulation of red blood cell production in a mammal comprising:
(i) identifying a mammal having a cancer that expresses or is suspected of expressing a receptor for erythropoietin;
(ii) introducing into said mammal a population of genetically engineered hematopoietic cells, said population of cells comprising a recombinant nucleic acid construct encoding a fusion protein comprising at least one signaling domain and at least one ligand binding domain which is heterologous with respect to the signaling domain, wherein binding of a ligand to the ligand binding domain results in the dimerization or oligomerization of at least two of said fusion proteins thereby activating the signaling domain and initiating a signal for proliferation or differentiation of said population of cells, wherein the proliferation or differentiation of said population of genetically engineered hematopoietic cells results in an increase in red blood cell levels in said mammal; and
(iii) contacting said hematopoietic cells with a ligand, wherein said ligand binds to the ligand binding domain.

20. The method of claim 19, further comprising providing an anti-cancer agent to the mammal.

21. A method for the regulation of red blood cell production in a mammal comprising:
(i) identifying a mammal having a cancer that expresses or is suspected of expressing a receptor for erythropoietin;
(ii) introducing into one or more hematopoietic cells at least one recombinant nucleic acid construct encoding a fusion protein comprising at least one signaling domain and at least one ligand binding domain which is heterologous with respect to the signaling domain, wherein binding of a ligand to the ligand binding domain results in the dimerization or oligomerization of at least two of said fusion proteins thereby activating the signaling domain and initiating a signal for proliferation or differentiation of said cells, wherein the proliferation or differentiation of said population of genetically engineered hematopoietic cells results in an increase in red blood cell levels in said mammal; and
(iii) contacting said hematopoietic cells with a ligand, wherein said ligand binds to the ligand binding domain.

22. The method of claim 21, further comprising providing an anti-cancer agent to the mammal.
